(19) United States
(12) Patent Application Publication
(10) Pub. No.: US 2017/0349953 A1
(43) Pub. Date: Dec. 7, 2017

(54) NOVEL MUTATIONS IN ANAPLASTIC LYMPHOMA KINASE PREDICTING RESPONSE TO ALK INHIBITOR THERAPY IN LUNG CANCER PATIENTS

(71) Applicant: Roche Sequencing Solutions, Inc., Pleasanton, CA (US)

(72) Inventors: Alexander Lovejoy, Newark, CA (US); Stephanie Yaung, San Jose, CA (US); Aarthi Balasubramanyam, Sunnyvale, CA (US); John Palma, Alamo, CA (US)

(21) Appl. No.: 15/611,612

(22) Filed: Jun. 1, 2017

Related U.S. Application Data
(60) Provisional application No. 62/344,297, filed on Jun. 1, 2016.

(51) Int. Cl. C12Q 1/68; A61K 39/00

(52) U.S. Cl. CPC C12Q 1/6886; A61K 39/0011; C12Q 2600/158; C12Q 2600/106; A61K 2123/00; C12Q 2600/172; C12Q 2600/156

(57) ABSTRACT

The invention comprises novel methods and compositions for detecting whether a patient will be responsive to ALK inhibitors and methods of treating the patient.

### CROSS-REFERENCES TO RELATED APPLICATIONS

The present application claims priority to U.S. Provisional Application 62/344,297 filed Jun. 1, 2016, the disclosure of which is incorporated herein by reference.

### FIELD OF THE INVENTION

The present disclosure relates to cancer diagnostics and companion diagnostics for cancer therapies. In particular, the invention relates to the detection of mutations that are useful for diagnosis and prognosis as well as predicting the effectiveness of treatment of cancer.

### BACKGROUND OF THE INVENTION

Gene activation via fusions in the introns of Anaplastic Lymphoma Kinase (ALK) are a common genomic driver of non-small cell lung cancer (NSCLC). In lung cancer patients where ALK fusions are detected, targeted anti- ALK therapy can be prescribed. For example, the drug crizotinib (XALKORI®) is an inhibitor of among others, the ALK protein. Crizotinib has been shown to significantly improve progression-free survival of patients with ALK fusions. However, patients do inevitably progress after being given crizotinib, due at least in part to the emergence of resistance mutations. The missense mutations known so far are L1152R, C1156Y, F1174L, L1196M, G1269A, and G1548E (reviewed in Van der Wekken et al. (2016) Crit. Rev. Onc. Hematol. 100:107.).

There are second-line therapies which are effective in prolonging progression-free survival of patients who have progressed on first--line therapy. However, patients respond to these second-line therapies to different extents. Knowing the genetic basis for which patients respond well to second-line therapies and which patients do not would greatly help selection of treatment for patients that progress on the first-line therapy.

Some mutations in ALK are common, while others occur less frequently. Ideally, a clinical test for ALK mutations targets as many mutations as possible. This will assure that patients with rare mutations do not receive a “false negative” test result. If a rare mutation goes undetected, the patient with such a mutation will not receive an optimal treatment plan and may be given an ineffective medication for his or her tumor. Therefore when a new mutation in the ALK gene is discovered, detecting this mutation has the potential of affecting the clinical outcome in some patients.

### BRIEF DESCRIPTION OF THE DRAWINGS

FIG. 1 shows detection of the R1209Q mutation and mutation frequencies in patient samples before and after alectinib treatment. The mutation was detected in five of the six patients after alectinib treatment.

FIG. 2 shows different mutations found in the sixth patient before and after alectinib treatment.

FIG. 3 shows Progression Free Survival (PFS) of patients with and without ALK resistance (ALKR) mutations. Months of PFS are indicated on the x-axis. Those without an ALK resistance mutation have significantly longer PFS,

FIG. 4 shows Overall Survival (OS) of patients with and without ALK resistance mutations. Months of OS are indicated on the x-axis. Those without an ALK resistance mutation have significantly longer OS.

FIG. 5 shows PFS of patients with or without ALK resistance (ALKR) mutations, and with or without ALK fusions (ALK). Patients with neither ALKR nor an ALK fusion had the longest PFS, followed by those with an ALK fusion and no ALKR, followed by those with an ALK fusion and an ALKR mutation.

FIG. 6 shows OS of patients with or without ALK resistance (ALKR) mutations, and with or without ALK fusions (ALK). Patients with neither ALKR nor an ALK fusion had the longest OS, followed by those with an ALK fusion and no ALKR, followed by those with an ALK fusion and an ALKR mutation.

FIG. 7 shows the effect of more ALK and non-ALK mutations on PFS. Patients with 4 or fewer mutations had a longer PFS than patients with more than 4 mutations.

FIG. 8 shows the effect of variant 3 ALK fusion compared to other ALK fusions on progression free survival time. PFS is significantly lower for patients with a variant 3 ALK fusion compared to that of patients with a non-variant 3 ALK fusion.

### SUMMARY OF THE INVENTION

Provided herein is a method of treating a patient having a tumor possibly harboring cells with a mutation in anaplastic lymphoma kinase (ALK) gene, comprising: testing a sample from the patient for the presence of at least one of the mutations R1209Q (G3636A) and I1268V (A3802G). If the mutation R1209Q is present, the method comprises not administering to the patient an ALK inhibitor compound, or if the patient is receiving an ALK inhibitor compound, administering an alternative ALK inhibitor compound or not administering an ALK inhibitor compound. If the mutation I1268V is present or no mutations are present, the method comprises administering to the patient an ALK inhibitor compound. In some embodiments, the ALK inhibitor compound can be alectinib, crizotinib, ceritinib, brigatinib, lorlatnib, or entrectinib. In some embodiments, the testing is performed using an oligonucleotide complementary to the mutant sequence, for example, the testing may be performed by allele-specific PCR or PCR (e.g., qPCR or real time PCR) with an allele-specific probe. In some embodiments, the method further comprise testing the sample for the presence of one or more ALK mutations G1202R, I1171T, V1180L, I1171N, 11171S, R1209Q, T1151, and G1548E and if any of the mutations is present, not administering to the patient the ALK inhibitor compound, or if the patient is receiving an ALK inhibitor compound, administering an alternative ALK inhibitor compound or not administering an ALK inhibitor compound. In some embodiments, the method further comprises testing the sample for the presence of one or more ALK mutations I1268V, 51206F, 51206Y, G1269A, L1196M, L1196Q, C1156Y, L1152R, F1174L, F1174C, and F1174V and if any of the mutations is present, administering an ALK inhibitor compound or alternative ALK inhibitor compound if treatment is ongoing.

In some embodiments, the method further comprises testing the sample for the presence of one or more ALK fusion products. In some embodiments, the ALK fusion product is an EML4-ALK fusion. In some embodiments, the ALK fusion product is an EML4-ALK fusion variant 3. If an ALK fusion product is present, the method further comprises administering an ALK inhibitor compound. If an ALK fusion product is detected as well as at least one ALK mutation selected from the group consisting of G1202R, I1171T, V1180L, I1171N, 111715, R1209Q, T1151, and G1548E, the method further comprises administering alternative therapy (e.g., alternative ALK inhibitor therapy) if the patient is receiving or has received an ALK inhibitor compound, or not administering an ALK inhibitor compound. If an ALK fusion product is detected as well as at least one ALK mutation selected from the group consisting of I1268V, 51206F, 51206Y, G1269A, L1196M, L1196Q, C1156Y, L1152R, F1174L, F1174C, and F1174V, the method further comprises administering ALK inhibitor therapy. In some embodiments, the ALK inhibitor compound or alternative ALK inhibitor compound is selected from alectinib, crizotinib, ceritinib, brigatinib, lorlatinib, or entrectinib.

In some embodiments, the sample from the patient indudes RNA and the one or more ALK mutation and/or one or more ALK fusion products is detected using reverse transcription PCR (RT-PCR) or Fluorescence In Situ Hybridization (FISH). In some embodiments, the sample indudes DNA and the one or more ALK mutation and/ or one or more ALK fusion products is detected using PCR or another nucleic amplification technique.

Further provided are methods for determining the likelihood of response of a cancer patient to ALK inhibitor therapy (e.g., ALK inhibitor compound) comprising: testing a sample from the patient for one or both ALK mutations R1209Q and I1268V. If the mutation R1209Q is present, the method comprises reporting that the patient likely will not respond to an ALK inhibitor compound or will not continue to respond to an ALK inhibitor compound that has been administered to the patient. If the mutation I1268V is present or no mutation is present, reporting that the patient will likely respond to an ALK inhibitor compound. In some embodiments, the ALK inhibitor compound is selected from the group consisting of crizotinib, ceritinib, alectinib, brigatinib, lorlatinib, and entrectinib. In some embodiments, the testing is performed using an oligonucleotide complementary to the mutant sequence, for example the testing may be performed by allele-specific PCR or PCR (e.g., qPCR or real time PCR) with an allele-specific probe. In some embodiments, the method further comprises testing the sample for the presence of one or more ALK mutations selected from the group consisting of G1202R, I1171T, V1180L, I1171N, I1171S, R1209Q, T1151, and G1548E. If any of the mutations is present, the method comprises reporting that the patient is not likely to respond to an ALK inhibitor compound, or is unlikely to continue responding to the same ALK inhibitor compound if the patient has received treatment with an ALK inhibitor compound. In some embodiments, the method further comprises testing the sample for the presence of one or more ALK fusion products. In some embodiments, the ALK fusion product is an EML4-ALK fusion. In some embodiments, the ALK fusion product is an EML4-ALK fusion variant 3. If an ALK fusion product is present, the method comprises reporting that the patient is likely to respond to an ALK inhibitor compound. If an ALK fusion product is detected as well as at least one ALK mutation selected from the group consisting of G1202R, I1171T, V1180L, I1171N, I1171S, R12090, T1151, and G1548E, the method comprises reporting that the patient may respond to an alternative therapy (e.g., alternative ALK inhibitor therapy) if the patient is receiving or has received an ALK inhibitor compound, or will not respond to an ALK inhibitor compound.

Also provided are methods of selecting anaplastic lymphoma kinase (ALK) inhibitor therapy for a patient having a tumor with an ALK mutation in the gene who has been previously treated with alectinib, comprising testing a sample from the patient for the presence of one or both ALK mutations R1209Q (G3636A) and I1268V (A3802G). If the R1209Q mutation is present the method comprises not selecting or not administering an ALK inhibitor compound, or selecting or administering an alternative ALK inhibitor compound. If the I1268V mutation is present or no mutations are present, selecting or administering alectinib for the patient. In some embodiments, the method comprises testing the sample for the presence of one or more ALK mutations selected from the group consisting of G1202R, I1171T, V1180L, I1171N, I1171S, R1209Q, T1151, and G1548E and if any of the mutations is present, not selecting or not administering to the patient an ALK inhibitor compound, or selecting or administering an alternative ALK inhibitor compound for the patient. In some embodiments, the alternative ALK inhibitor compound is selected from the group consisting of crizotinib, ceritinib, brigatinib, and entrectinib. In some embodiments, the method further comprises testing the sample for the presence of one or more ALK fusion products. In some embodiments, the ALK fusion product is an EML4-ALK fusion. In some embodiments, the ALK fusion product is an EML4-ALK fusion variant 3. If an ALK fusion product is present, the method comprises selecting or administering an ALK inhibitor compound for the patient. If an ALK fusion product is detected as well as at least one ALK mutation selected from the group consisting of G1202R, I1171T, V1180L, I1171N, I1171S, R1209Q, T1151, and G1548E, the method comprises selecting or administering no ALK inhibitor compound, or selecting or administering an alternative ALK inhibitor compound.

Also provided herein are kits for detecting mutations in the ALK gene comprising oligonucleotides for specific detection of the R1209Q ALK mutation, e.g., primers and at least one probe e.g., labeled probe). In some embodiments, the kit further comprises oligonucleotides for the specific detection of at least one ALK resistance mutation selected from the group consisting of G1202R, I1171T, V1180L, I1171N, I1171S, R1209Q, T1151, and G1548E. In some embodiments, the kit further comprises oligonudeotides for the specific detection of the I1268V ALK mutation. In some embodiments, the kit further comprises oligonudeotide for the detection of at least one ALK fusion product. In some embodiments, the at least one ALK fusion product includes an EML4-ALK fusion, e.g., EML4-ALK fusion variant 3. In some embodiments, the kits further comprise reagents for carrying out amplification and detection using the included oligonucleotides, e.g., nucleic acid polymerase(s), reverse transcriptase, cofactors, dNTPs, buffers, etc.

Also provided herein are kits for detecting mutations in the ALK gene comprising oligonucleotides for specific detection of the I1268V ALK mutation, e.g., primers and at least one probe (e.g., labeled probe). In some embodiments, the kit further comprises oligonucleotides for the specific detection of at least one ALK resistance mutation selected from the group consisting of G1202R, I1171T, V1180L, I1171N, I1171S, R1209Q, T1151, and G1548E. In some embodiments, the kit further comprises oligonudeotides for the specific detection of the R1209Q ALK mutation. In some embodiments, the kit further comprises oligonudeotide for the detection of at least one ALK fusion product. In some embodiments, the at least one ALK fusion product includes an EML4-ALK fusion, e.g., and EML4-ALK fusion variant 3. In some embodiments, the kits further comprise reagents for carrying out amplification and detection using the included oligonucleotides, e.g., nucleic acid polymerase(s), reverse transcriptase, cofactors, dNTPs, buffers, etc.

Further provided are methods for determining if a cancer patient will be responsive to an ALK inhibitor compound. In some embodiments, the method comprises (a) testing a sample from the patient for the presence of one or more ALK fusion products; (b) if one or more ALK fusion products are present, determining that the patient will be responsive to an ALK inhibitor compound; (c) testing the sample (or a different sample) from the patient for the presence of one or more ALK resistance mutations selected from the group consisting of G1202R, I1171T, V1180L, I1171N, I1171S, R12090, T1151, and G1548E; and (d) if one or more ALK resistance mutations is present, determining that the patient will not be responsive to an ALK inhibitor compound, or will not be responsive to the same ALK inhibitor compound if the patient has received ALK inhibitor compound therapy. If both the one or more ALK fusion products and one or more ALK resistance mutations are present, determining that the patient will not be responsive to an ALK inhibitor compound, or will not be responsive to the same ALK inhibitor compound if the patient has received ALK inhibitor compound therapy. In some embodiments, the method further comprises treating the patient according to the determination. That is, if an ALK fusion product is present, administering an ALK inhibitor compound to the patient; if an ALK resistance mutation is present, not administering an ALK inhibitor compound, or administering an alternative ALK inhibitor compound if the patient has received ALK inhibitor compound therapy; or if both an ALK fusion product and an ALK resistance mutation are present, not administering an ALK inhibitor compound, or administering an alternative ALK inhibitor compound if the patient has received ALK inhibitor compound therapy. In some embodiments, the ALK inhibitor compound or alternative ALK inhibitor compound is selected from the group consisting of alectinib, crizotinib, ceritinib, brigatinib, lorlatnib, and entrectinib. In some embodiments, the at least one ALK fusion product includes an EML4-ALK fusion.

Further included are methods for predicting prognosis in a patient with cancer, e.g., NSCLC. In some embodiments, the method comprises determining the presence or absence of a variant 3 EML4-ALK fusion product in a sample from the patient; predicting a worse prognosis (e.g., reduced progression free survival time, reduced overall survival time, more severe disease symptoms, etc.) for the patient if the presence of a variant 3 EML4-ALK fusion product is detected compared to the prognosis of a patient with a different (non-variant 3) ALK fusion product. In some embodiments, the determining is carried out by a method selected from the group consisting of NGS, PCR, FISH, and IHC (immunohistochemistry). In some embodiments, the sample is selected from the group consisting of blood or a blood product, and a tissue sample from the patient including tumor tissue (e.g., fresh tissue or FFPET sample).

Further included are methods for predicting prognosis in a patient with cancer, e.g., NSCLC. In some embodiments, the method comprises determining the number of mutations in a sample from the patient. In some embodiments, the method comprises determining the number of mutations in the patient sample; and predicting a worse prognosis (e.g., reduced progression free survival time, reduced overall survival time, more severe disease symptoms, etc.) for the patient if the patient sample includes more than 4 mutations compared to the prognosis of a patient with 4 or less mutations. In some embodiments, the determining is carried out by a method selected from the group consisting of NGS, PCR, FISH, and IHC (immunohistochemistry). In some embodiments, the sample is selected from the group consisting of blood or a blood product, and a tissue sample from the patient including tumor tissue (e.g., fresh tissue or FFPET sample). In some embodiments, the mutations detected are selected from cancer associated genes, e.g., ALK and other genes shown in Example 5 (Tables 2, 4, and 5). In some embodiments, mutations for at least 10, 20, 30, 40, 50, 60, 70, 80, 90, 100, 250, 500, 1000, or more genes are tested for mutation status.

### DETAILED DESCRIPTION OF THE INVENTION

### Definitions

The following definitions aid in understanding of this disclosure.

The terms “ALK resistant mutation,” “ALK resistance mutation,” “ALK inhibitor resistant mutation,” “ALK therapy resistant mutation” and like terms are used to refer to mutations in the ALK gene that confer resistance to ALK inhibition, e.g., alectinib. Examples of ALK resistant mutations include R1209Q, L1152R, C1156Y, F1174L, L1196M, G1269A, and G1548E.

The term “ALK fusion,” “ALK fusion product,” and like terms refer to gene fusion products involving ALK. Many of these fusion products result in abnormally high expression and/ or kinase activity of ALK. Examples include fusions of EML4, KIF5B, HIP1, KLC1, or TFG with ALK. Specific fusions are shown in Tables 2 and 5 herein.

As used herein the term “responsive to therapy,” “responsive to an inhibitor compound,” and like terms refers to a positive response to therapy or inhibitor compound by a cancer patient. The responsiveness can be increased progression free survival (PFS) or overall survival, reduced tumor size, reduced rate of tumor growth or metastasis, improved well-being, etc.

The term “allele-specific PCR” or “PCR with allele-specific primer” refer to PCR with a primer that hybridizes to more than one variant of the target sequence (e.g., wild-type and mutant variants), but is capable of discriminating between the variants of the target sequence in that only with one of the variants (e.g., the mutant variant), the primer is efficiently extended by the nucleic acid polymerase under suitable conditions. With other variants of the target sequence (e.g., wild type), the extension is less efficient, inefficient or undetectable.

The terms “sample,” “sample from a patient,” patient sample,” and like terms refer to any composition containing or presumed to contain target nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). A sample can also refer to processed tissue or biological fluid, e.g., purified or partially purified nucleic acids.

The terms “nucleic acid,” “polynucleotide,” and “oligonucleotide” can be used interchangeably to refer to a multimer or polymer of single nucleotides. “Oligonucleotide” is a term sometimes used to describe a shorter polynucleotide. An oligonucleotide may be comprised of at least 6 nucleotides, for example at least about 10-12 nucleotides, or at least about 15-30 nucleotides, e.g., corresponding to a region of the designated nucleotide sequence. The term “nucleotide” typically refers to a monomer or single base.

The term “primer” refers to an oligonucleotide which hybridizes with a sequence in the target nucleic acid and is capable of acting as a point of initiation of synthesis along a complementary strand of nucleic acid under conditions suitable for such synthesis. A forward primer and reverse primer set the boundaries of an amplicon and produce an amplification product when exposed to a nucleic acid polymerase under appropriate conditions. As used herein, the term “probe” refers to an oligonucleotide which hybridizes with a sequence in the target nucleic acid and is usually detectably labeled. The probe can have modifications, such as a 3′-terminus modification that makes the probe non-extendable by nucleic acid polymerases, and one or more non-naturally occurring labels, e.g., fluorophore, chromophore, optionally in combination with a quencher. An oligonucleotide with the same sequence may serve as a primer in one assay and a probe in a different assay.

As used herein, the terms “target sequence”, “target nucleic acid” or “target” refer to a portion of the nucleic acid sequence in the sample which is to be detected or analyzed. The term target includes all variants of the target sequence, e.g., one or more mutant variants and the wild type variant.

The term “sequencing” refers to any method of determining the sequence of nucleotides in the target nucleic acid.

The terms “patient” and “subject” refer to an individual that may or may not be diagnosed with or treated for a disease, but is the subject of medical care.

The terms “administer,” “administering,” and like terms are not limited to physical administration, but include recommending or prescribing a therapeutic regimen (e.g., drug or chemotherapeutic treatment).

### Methods for Detecting ALK Mutations and Fusion Variants, Prognosis, and Treatment

Novel mutations in the kinase domain of ALK that are useful for cancer diagnosis and prognosis, as well as a designing a therapy regimen and predicting success of the therapy regimen are provided herein. Moreover, the effect of multiple abnormalities in the ALK gene (e.g., ALK fusion products and ALK resistance mutations) on prognosis and therapeutic efficacy is described herein.

Abnormal activation of ALK is known to drive several types of cancer. Approximately 60% of anaplastic lymphomas and 3-5% of non-small cell lung cancers (NSCLC) have ALK activated through gene fusions and mutations. ALK has also been found to be abnormally active neuroblastomas, glioblastomas, esophageal and breast cancers. Abnormal activation of ALK often involves a gene fusion, most commonly EML4-ALK (echinoderm microtubule-associated protein like 4-anaplastic lymphoma kinase). EML4-ALK fusions are associated with non-small cell lung cancer (NSCLC). In the case of most fusions, the N terminal, extracellular portion of ALK is replaced by EML4 (or KIF5B, HIP1, KLC1, or TFG). The expression of the resulting fusion gene is driven by a strong promoter, e.g., the EML promoter, resulting in higher expression of the intracellular tyrosine kinase domain of ALK. In addition, EML4 forms a coiled-coil that results in ligand-independent dimerization, and constitutive activation of the ALK tyrosine kinase domain

Several small-molecule inhibitors of the ALK kinase are currently on the market or in clinical trials. These include crizotinib (XALKORI®), ceritinib (ZYKADIA®), alectinib (ALECENSA®), brigatinib, lorlatinib, entrectinib and other compounds that are currently in early stages of development. Analysis of clinical outcomes as well as in vitro cell line studies revealed that resistance to ALK inhibitors often develops as a result of missense mutations in the ALK gene (reviewed in Van der Wekken et al. (2016) Crit. Rev. Onc. Hematol. 100:107.)

Described herein are novel variants R1209Q (G3626A) and I1268V (A3802G) in the ALK gene discovered in cancer patients undergoing ALK inhibitor therapy. In the reference human genome hg19, R1209Q corresponds to chr2:29443591:C>T and I1268V correspond to chr2:29432686:T>C. In the reference human genome hg38, R1209Q corresponds to chr2:29220725:C>T and I1268V corresponds to chr2:29209820:T>C.

In most patients, the variant R1209Q is detected after the ALK inhibitor therapy was administered, providing evidence that the mutation may confer resistance to therapy. The mutation was, however, present both before and after the ALK inhibitor therapy was administered in one patient studied. The other variant, I1268V, was identified in patients only before the therapy was administered suggesting that it may confer sensitivity to the therapy,

Mutant ALK gene or gene product (i.e., mutant mRNA or mutant protein) can be detected in tumor tissue (e.g., fresh or FFPET tissue), bronchoaveolar lavage, or other body samples such as urine, sputum, plasma, or serum where tumor cells or tumor nucleic acids may be present. The mutations can also be detected in cell-free material where cell-free tumor DNA or RNA may be present, e.g., urine, sputum, plasma, or serum.

Methods for isolating nucleic acids from biological samples are known, e.g., as described in Sambrook et al., MOLECULAR CLONING, A LABORATORY MANUAL, Cold Springs Harbor Press (Cold Springs Harbor, N.Y. 1989), and several kits are commercially available (e.g., High Pure RNA Isolation Kit, High Pure Viral Nucleic Acid Kit, and MagNA Pure LC Total Nucleic Acid Isolation Kit from Roche). In some embodiments, DNA is prepared, and used as template for the presently disclosed amplification and detection methods. In some embodiments, RNA Is prepared. When RNA is used as template for amplification by PCR, a reverse transcription step is required to prepare cDNA. A DNA polymerase such as Taq, Taq derivatives, or other thermostable polymerases can then be used to carry out amplification.

Provided herein are methods of detecting mutations R1209Q (G3626A) and I1268V (A3802G) in ALK gene by allele-specific PCR with mutation-specific oligonucleotide primers (e.g., allele-specific primer). An allele-specific primer typically possesses a 3′ end matched to the target sequence (e.g., the mutant sequence) and mismatched to the alternative sequence (e.g., the wild-type sequence). Optionally, allele-specific primers may contain internal mismatches with both the wild-type and mutant target sequence. Additional mismatches in allele-specific PCR primers have been shown to increase selectivity of the primers. See U.S. Pat. No. 8,586,299.

In some embodiments, the method further comprises using allele-specific PCR to detect one or more ALK mutations G1202R, I1171T, V1180L, I1171N, I1171S, R1209Q, T1151, G1548E, I1268V, 51206F, S1206Y, G1269A, L1196M, L1196Q, C1156Y, L1152R, F1174L, F1174C, and F1174V in the patient sample in any combination. In some embodiments, the method further comprises determining if a sample from a patient includes at least one ALK fusion product (see, e.g., Table 2), e.g., in RNA from the sample. Fusion products can be detected using amplification primers on either side of the fusion point (e.g., one primer complementary to ALK sequence and the other primer complementary to sequence from the fusion partner, e.g., EML4) or with one primer that is complementary to sequence encompassing the fusion point of a particular fusion product. In some embodiments, a fusion is detected using a labeled probe that is complementary (hybridizes) to sequence encompassing the fusion point of a particular fusion product. In some embodiments, the labeled probe is complementary to sequence in ALK or its fusion partner, and a fusion is detected based on the size or presence of the amplification product that hybridizes to the probe.

Further provided are methods for detecting mutations R1209C) (G3626A) and I1268V (A3802G) in ALK gene with a specific probe. The probe may be used in a number of nucleic acid detection technologies, e.g., Southern or Northern hybridization, real-time PCR, or NGS. A typical mutation-specific detection probe forms a stable hybrid with the target sequence (e.g., the mutant sequence) and does not form a stable hybrid with the alternative sequence (e.g., the wild-type sequence at the same site) under the reaction conditions at which the detection is carried out. For successful probe hybridization, the probe needs to have at least partial complementarity to the target sequence. Generally, complementarity close to the central portion of the probe is more critical than complementarity at the ends of the probe. See, e.g., Innis et al., Academic Press, NY, 1990 Chapter 32, pp. 262-267. In some embodiments, the probe has a particular structure, including a protein-nucleic acid (PNA), a locked nucleic acid (LNA), a molecular beacon probe (Tyagi et al. (1996) Nat. Biotechnol. 3:303-308) or can be included in SCORPIONS® self-probing primers (Whitcombe et al. (1999) Nat. Biotechnol. 8:804-807). A probe can be labeled with a radioactive, a fluorescent or a chromophore label, optionally in combination with a quencher moiety, e.g., BHQ. For example, the mutations may be detected by real-time allele-specific polymerase chain reaction, where hybridization of a probe to the amplification product results in enzymatic digestion of the probe and detection of the digestion products (TaqMan probe, Holland et al., (1991) P.N.A.S. USA 88:7276-7280). Hybridization between the probe and the target may also be detected by detecting a change in fluorescence due to the nucleic acid duplex formation. (U.S. application Ser. No. 12/330,694, filed on Dec. 9, 2008) or by detecting the characteristic melting temperature of the hybrid between the probe and the target (U.S. Pat. No. 5,871,908). In some embodiments, the method further comprises using hybridization probes to detect one or more ALK mutations G1202R, I1171T, V1180L, I1171N, I1171S, R1209Q, T1151, G1548E, I1268V, S1206F, S1206Y, G1269A, L1196M, L1196Q, C1156Y, L1152R, F1174L, F1174C, and F1174V in the patient sample in any combination. In some embodiments, the method further comprises determining if a sample from a patient includes at least one ALK fusion product (see, e.g., Table 2), e.g., in RNA from the sample.

Provided herein are methods of treating a patient having a tumor possibly harboring cells with a mutant ALK gene with an ALK inhibitor, the method comprising testing the patient sample for the presence of one or both ALK mutations R1209Q (G3626A) and I1268V (A3802G). If the mutation I1268V is found, the method further comprises administering ALK inhibitor therapy (e.g., ALK inhibitor compound such as crizotinib, ceritinib, alectinib, brigatinib, lorlatinib, or entrectinib) but if the mutation R1209Q is found, the method further comprises administering alternative therapy (e.g., alternative ALK inhibitor therapy) if ALK inhibitor therapy is already ongoing, or not administering the ALK inhibitor therapy. In some embodiments, the method further comprises testing the patient sample for the presence of one or more ALK mutations selected from the group consisting of: G1202R, I1171T, V1180L, I1171N, I1171S, T1151, and G1548E (e.g., any 1, 2, 3, 4, 5, 6, 7, of the ALK resistance mutations in any combination, or all 8 ALK resistance mutations), and if at least one of those mutations is found, the method further comprises administering alternative therapy (e.g., alternative ALK inhibitory therapy) if ALK inhibitory therapy is ongoing, or not administering the ALK inhibitor therapy. In some embodiments, the method further comprises testing the patient sample for the presence of one or more ALK mutations selected from the group consisting of S1206F, S1206Y, G1269A, L1196M, L1196Q, C1156Y, L1152R, F1174L, F1174C, and F1174V (e.g., any 1, 2, 3, 4, 5, 6, 7, 8, 9, 10 of the ALK susceptibility mutations in any combination, or all 11 susceptibility mutations), and if at least one susceptibility mutation is detected, administering ALK inhibitor therapy (e.g., an alternative ALK inhibitor if treatment is ongoing). In some embodiments, the method further comprises determining if a sample from a patient includes at least one ALK fusion product. In the event an ALK fusion product is detected, the method further comprises administering ALK inhibitor therapy. In the event an ALK fusion product is detected as well as at least one ALK mutation selected from the group consisting of: G1202R, I1171T, V1180L, I1171N, I1171S, R1209Q, T1151, and G1548E (e.g., any 1, 2, 3, 4, 5, 6, 7, of the ALK resistance mutations in any combination, or all 8 ALK resistance mutations), the method further comprises administering alternative therapy (e.g., alternative ALK inhibitor therapy) in the event ALK inhibitor therapy is ongoing, or not administering ALK inhibitor therapy. In some embodiments, the method further comprises testing the patient sample for the presence of one or more ALK mutations selected from the group consisting of S1206F, S1206Y, G1269A, L1196M, L1196Q, C1156Y, L1152R, F1174L, F1174C, and F1174V (e.g., any 1, 2, 3, 4, 5, 6, 7, 8, 9, 10 of the ALK susceptibility mutations in any combination, or all 11 susceptibility mutations), and if at least one susceptibility mutation is detected, administering ALK inhibitor therapy (e.g., an alternative ALK inhibitor if treatment is ongoing). In some embodiments, the ALK inhibitor therapy (e.g., ALK inhibitor compound) or alternative ALK inhibitor therapy is selected from alectinib, crizotinib, ceritinib, brigatinib, lorlatinib, or entrectinib.

Multiple mutations can be detected simultaneously or separately by using hybridization to multiple probes, for example in a dot-blot or nucleic acid array format, multiplex PCR, for example multiplex allele-specific PCR and multiplex PCR followed by a probe melting assay with each probe characterized by a mutation-specific melting temperature. Multiple mutations may also be detected by nucleic acid sequencing. Sequencing can be performed by any method known in the art. Especially advantageous is the high-throughput single molecule sequencing (next generation sequencing, or NGS). Examples of such technologies include Illumina HiSeq platform (Illumina, San Diego, Calif.), Ion Torrent platform (Life Technologies, Grand Island, N.Y.), Pacific BioSciences platform utilizing the SMRT® reagents (Pacific Biosciences, Menlo Park, Calif.), or nanopore-based sequencing technology developed by Genia Technologies (Roche Genia, Santa Clara, Calif.) or Oxford Nanopore Technologies (Cambridge, UK) or any other presently existing or future single-molecule sequencing technology that does or does not involve sequencing by synthesis. Fusions can be detected by designing primers or probes specific for particular ALK fusions, or that can detect the presence of more than one ALK fusion, e.g., as described in U.S. Pat. No. 7,700,339 and US20160304937.

The sequencing technology may include a data analysis step that is able to increase sensitivity and specificity of detecting very small amounts of target nucleic acid, e.g., from circulating tumor DNA (ctDNA) present in a patient's blood serum in very small amounts. Examples of such methods induding sample barcoding and error correction are described in U.S. patent applications US20140296081 and US20160032396.

Further provided are methods for determining a likelihood of response of a malignant tumor in a patient to ALK inhibitors. In some embodiments, the method comprises testing a sample from the patient for the presence of one or both of the mutations R1209Q (G3626A) and I1268V (A3802G). If the mutation I1268V is found, the method comprises reporting that the patient is likely to respond to ALK inhibitor therapy (e.g., ALK inhibitor compound). If the mutation R1209Q is found, the method comprises reporting that the patient is not likely to respond to the ALK inhibitor therapy, in particular if the patient is being treated with an ALK inhibitor. In such cases, the method comprises reporting that the patient is unlikely to respond to the same ALK inhibitor therapy, but that the patient may respond to alternative therapy, induding alternative ALK inhibitor therapy. In some embodiments, the method further comprises testing the sample from the patient for the presence of one or more ALK resistance mutations selected from L1152R, C1156Y, F1174L, L1196M, G1269A, and G1548E and if at least one of the ALK resistance mutations is found, reporting that the patient is not likely to respond to ALK inhibitor therapy, in particular if the patient is being treated with an ALK inhibitor. In such cases, the method comprises reporting that the patient is unlikely to respond to the same ALK inhibitor therapy, but that the patient may respond to alternative therapy, including alternative ALK inhibitor therapy. In some embodiments, the ALK inhibitor therapy is selected from alectinib, crizotinib, ceritinib brigati ib, lorlatinib, or entrectinib. In some embodiments, the method further comprises determining if a sample from a patient includes at least one ALK fusion product. In the event an ALK fusion product is detected, the method further comprises administering ALK inhibitor therapy. In the event an ALK fusion product is detected as well as at least one ALK resistance mutation selected from the group consisting of R1209, L1152R, C1156Y, F1174L, L1196M, G1269A, and G1548E, the method further comprises administering alternative therapy (e.g., alternative ALK inhibitor therapy) in the event ALK inhibitor therapy is ongoing, or not administering ALK inhibitor therapy.

Further provided are methods for selecting an ALK inhibitor for a patient with a malignant tumor who has been treated with alectinib. The method comprises testing a sample from the patient for the presence of one or both of the mutations R1209Q (G3626A) and I1268V (A3802G). If the mutation I1268V is found, the method comprises selecting alectinib as the ALK inhibitor therapy. If the mutation R1209Q is found, the method comprises selecting alternative therapy (e.g., alternative ALK inhibitor therapy) or no ALK inhibitor therapy. In some embodiments, the method further comprises testing the sample from the patient for the presence of one or more ALK mutations selected from L1152R, C1156Y, F1174L, L1196M, G1269A, and G1548E and if at least one of the mutations is found, selecting alternative therapy (e.g., alternative ALK inhibitor therapy) or no ALK inhibitor therapy. In some embodiments, the method further comprises determining if a sample from a patient includes at least one ALK fusion product. In the event an ALK fusion product is detected, the method further comprises administering ALK inhibitor therapy, e.g., including alectinib. In the event an ALK fusion product is detected as well as at least one ALK resistance mutation selected from the group consisting of R1209, L1152R, C1156Y, F1174L, L1196M, G1269A, and G1548E, the method further comprises administering alternative therapy (e.g., alternative ALK inhibitor therapy) or not administering ALK inhibitor therapy. In some embodiments, the alternative ALK inhibitor therapy is selected from crizotinib, ceritinib, brigatinib, lorlatinib, or entrectinib.

Provided herein are kits containing reagents necessary for detecting one or both of the mutations R1209Q (G3626A) and I1268V (A3802G) in the ALK gene. In some embodiments, the kit comprises oligonucleotides such as probes and amplification primers specific for the mutated sequences (i.e., able to distinguish wild type sequence from the mutated sequences) or capture probes for capturing the portions of the ALK gene where mutations R1209Q and I1268V are located.. In some embodiments, the kit contains reagents necessary for detecting mutations R1209Q (G3626A) and I1268V (A3802G) in DNA or the corresponding mRNA sequence. For example, the kit further comprises reagents necessary for the performance of amplification and detection assay, such as the components of PCR, real-time PCR, quantitative PCR, reverse transcription (e.g., for RT-PCR), and/or transcription mediated amplification (TMA). In some embodiments, the mutation-specific oligonucleotide is detectably labeled. In some embodiments, the kit comprises reagents for labeling and detecting the label. For example, if the oligonucleotide is labeled with biotin, the kit may comprise a streptavidin reagent with an enzyme and its chromogenic substrate. In some embodiments, the kit further includes reagents for detecting at least one more mutation in the ALK gene selected from the group consisting of G1202R, I1171T, V1180L, I1171N, I1171S, R1209Q, T1151, and G1548E (e.g., any 1, 2, 3, 4, 5, 6, 7, of the ALK resistance mutations in any combination, or all 8 ALK resistance mutations). In some embodiments, the kit further includes reagents for detecting at least one more mutation in the ALK gene selected from the group consisting of I1268V, S1206F, S1206Y, G1269A, L1196M, L1196Q, C1156Y, L1152R, F1174L, F1174C, and F1174V (e.g., any 1, 2, 3, 4, 5, 6, 7, 8, 9, 10 of the ALK susceptibility mutations in any combination, or all 11 susceptibility mutations).

In some embodiments, the kit comprises reagents for detecting mutations R1209Q (G3626A) and I1268V (A3802G) in mRNA. This embodiment shares elements with the kit for detecting the mutations in DNA and further comprises reagents for RNA- based detection including one or more of the following: a DNA polymerase with reverse transcriptase activity or a reverse transcriptase, an enzyme with RN Ase H activity and an oligo-dT capture reagent.

In some embodiments, the kit comprises reagents for detecting mutations R1209Q and I1268V in the ALK protein. The kit may comprise antibodies specific to the mutant ALK protein but not wild-type ALK protein, in some embodiments, the kit contains reagents for detecting the mutant protein in a blood (e.g., plasma or serum) sample from a patient. In some embodiments, the kit includes reagents for detecting the mutant in a tissue sample from a patient.

In some embodiments, kits are provided for detecting the presence of at least one ALK mutation or ALK fusion. In some embodiments, the kit includes oligonucleotides (e.g., primers and probes, or variants thereof such as Scorpion probes) for specifically detecting the ALK mutation R1209Q (i.e., able to distinguish the mutant sequence from, e.g., wild type sequence at the nucleotide positions encoding amino acid 1209). In some embodiments, the kit includes oligonucleotides for specifically detecting the ALK mutation I1268V. In some embodiments, the kit includes oligonucleotides for specifically detecting at least one ALK resistance mutation selected from the group consisting of: G1202R, I1171T, V1180L, I1171N, I1171S, R1209Q, T1151, and G1548E (e.g., any 1, 2, 3, 4, 5, 6, 7, of the ALK resistance mutations in any combination, or all 8 ALK resistance mutations). In some embodiments, the kit further includes reagents for detecting at least one more mutation in the ALK gene selected from the group consisting of I1268V, S1206F, S1206Y, G1269A, L1196M, L1196Q, C1156Y, L1152R, F1174L, F1174C, and F1174V (e.g., any 1, 2, 3, 4, 5, 6, 7, 8, 9, 10 of the ALK susceptibility mutations in any combination, or all 11 susceptibility mutations).

In some embodiments, the kit includes oligonucleotides for detecting at least one ALK fusion product. For example, the kit can include primers that fall on either side of the fusion point of one or more ALK fusion products, and probes that specifically detect individual fusion products, or that detect more than one fusion product. In some embodiments, the kit includes oligonucleotides for specifically detecting at least one ALK resistance mutation selected from the group consisting of: G1202R, I1171T, V1180L, I1171N, I1171S, R1209Q, T1151, and G1548E (e.g., any 1, 2, 3, 4, 5, 6, 7, of the ALK resistance mutations in any combination, or all 8 ALK resistance mutations), and one or more ALK fusion products. In some embodiments, the kit further includes reagents for detecting at least one more mutation in the ALK gene selected from the group consisting of I1268V, S1206F, S1206Y, G1269A, L1196M, L1196Q, C1156Y, L1152R, F1174L, F1174C, and F1174V (e.g., any 1, 2, 3, 4, 5, 6, 7, 8, 9, 10 of the ALK susceptibility mutations in any combination, or all 11 susceptibility mutations). In some embodiments, the one or more ALK fusion products can be selected from one or more of EML4-ALK (e.g., those listed in Table 2), KIF5B-ALK, HIP1-ALK, KLC1-ALK, and TFG-ALK.

In some embodiments, the kit further includes a thermostable DNA polymerase, reverse transcriptase, an enzyme with both activities, and/or any cofactors necessary for activity of the enzyme(s). In some embodiments, the kit further provides additional reagents that can be used in nucleic acid amplification, e.g., dNTPs and/or buffer reagents. In some embodiments, the kit further includes disposable components such as tubes, multiwell plates or capillary chips, etc.

In some embodiments, the kit further comprises primers and at least one probe for detecting an internal control in a sample from a patient, e.g., a housekeeping gene. In some embodiments, the kit further comprises at least one positive control, e.g., for each ALK mutation and ALK fusion detectable by the kit components. In some embodiments, the kit further comprises a negative control, e.g., wild type ALK human DNA or RNA.

### EXAMPLES

### Example 1

### Detecting ALK mutations in lung cancer patients

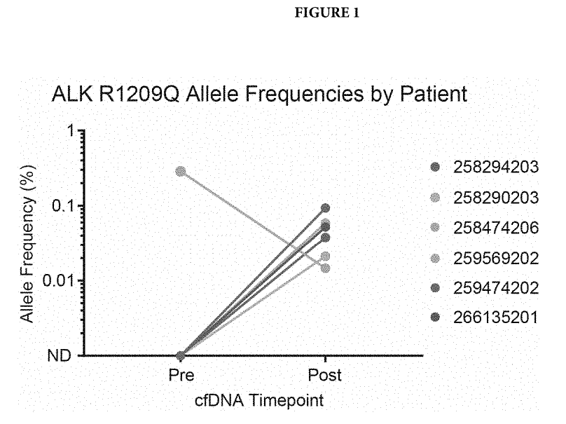

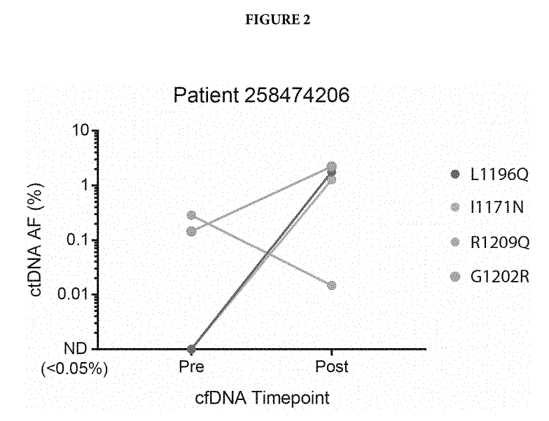

Cell free DNA from patients was isolated before alecitnib treatment and after alectinib treatment. This cfDNA was subjected to next-generation sequencing using the standard Illumina HiSeq workflow and analysis as described in US20140296081. Patient's single nucleotide variations (SNVs) were identified using the sequencing data, and SNVs that were present in only one of the two time points for a given patient were examined further. One variant, R1209Q, was identified in 6 patients. In 5/6 patients, it was only present after alectinib therapy (FIG. 1), providing evidence that it may confer resistance to alectinib. In the sixth patient, known resistance variants to alectinib were detected, so a separate clone may have conferred alectinib resistance (FIG. 2). The other variant, I1268V, was identified in one patient, but only before alectinib treatment, suggesting it may confer sensitivity to alectinib.

FIG. 1 shows detection of mutations and mutation frequencies in patient samples before and after alectinib treatment. One variant, R1209Q, was identified in 6 patients. In 5/6 patients, it was only present after alectinib therapy, providing evidence that it may confer resistance to alectinib. The other variant, I1268V, was identified in one patient, but only before alectinib treatment, suggesting it may confer sensitivity to alectinib.

FIG. 2 shows different mutations found in the sixth patient before and after alectinib treatment. In this patient, known resistance variants to alectinib were detected, so a separate clone may have conferred alectinib resistance.

### Example 2

### Factors Affecting ALK Fusion Outcomes

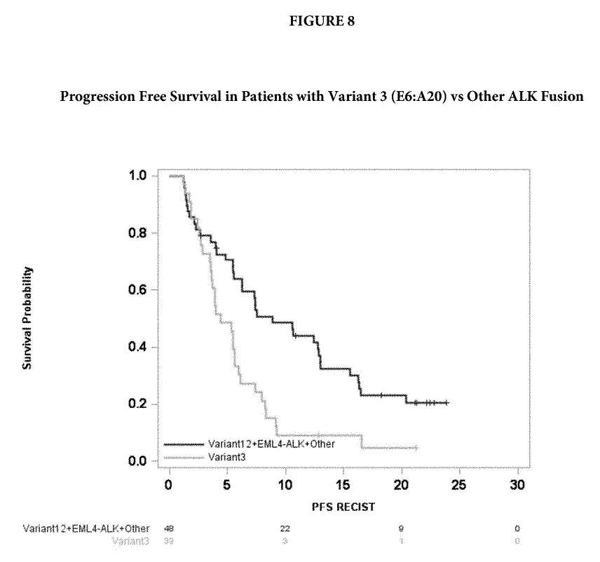

Table 1 shows that the hazard ratio for patients with the ALK fusion variant 3 (EML4 exon 6 joined to ALK exon 20) is much higher than for those without, FIG. 8 shows that the Progression Free Survival (PFS) is significantly shorter for patients with Variant 3 fusions versus other ALK fusions. This was found to be true regardless of race and treatment status individual with ALK fusion variant 3 oar ALK inhibitor treatment would have a worse outcome than an individual with a different ALK fusion on ALK inhibitor treatment). On average, one is about 2.6 times more likely to progress with the variant 3 fusion (p value 0.0012). This hazard ratio of 2.6 was from a Cox PH Multivariable Model performed on 72 patient plasma samples taken prior to treatment with alectinib. The model predicted progression free survival (PFS) from the variant 3 fusion effect, adjusting for confounders (race, baseline tumor measurement, etc). The hazard ratios are based on a value of 1 for Asian race. For example, a white individual with an ALK fusion has a 2.091-fold higher risk than an Asian individual with an ALK fusion.

TABLE 1

| Parameter | Hazard Ratio | Pr > ChiSq |
|---|---|---|
| Black or African American | 72.774 | 0.0004 |
| Multiple Races | 26.263 | 0.0037 |
| Other Race | 2.141 | 0.2490 |
| Unknown Race | 6.623 | 0.0202 |
| White | 2.091 | 0.0272 |
| Sum of longest diameter | 1.085 | 0.0148 |
| Variant 3 | 2.644 | 0.0012 |

### Example 3

### Single Nucleotide Variation (SNV) and ALK Fusion Analysis

Plasma samples were collected from 188 Stage IIIB-IV NSCLC (non-small cell lung cancer) patients who had progressed after crizotinib treatment (prior to 2^{nd} line treatment, e.g., with alectinib). These patients had been previously determined ALK-fusion positive by fluorescence in situ hybridization (FISH). The presence or absence of the most common ALK fusions was detected using a circulating tumor DNA panel (Avenio ctDNA panel). Table 2 shows the frequency of the detected fusions.

TABLE 2

| Fusion variant | Number |
|---|---|
| EML4 exon 13-ALK exon 20 | 26 |
| EML4 exon 13-ALK exon 24 | 1 |
| EML4 exon 14-ALK exon 20 | 1 |
| EML4 exon 18-ALK exon 20 | 3 |
| EML4 exon 19-ALK exon 20 | 1 |
| EML4 exon 20-ALK exon 20 | 4 |
| EML4 exon 21-ALK exon 20 | 3 |
| EML4 exon 6-ALK exon 20 | 33 |
| None detected | 107 |
| Other fusion variants | 9 |

The presence of ALK resistance mutations and ALK fusions in the samples was correlated as shown in Table 3. The ALK resistance mutations include G1202R, I1171T, V1180L, I1171N, I1171S, R1209Q, L1152R, C1156Y, F1174L, L1196M, G1269A, and G1548E and ALK fusions include those disclosed in Table 2. The results show that resistance mutations arise in ALK fusion variants.

TABLE 3

| | ALK Resistant Mutation (ALKR) | | |
|---|---|---|---|
| ALK Fusion Variant (ALK) | Mutated | Not mutated | Total |
| Variant | 11 | 70 | 81 |
| Non-variant | 0 | 107 | 107 |
| Total | 11 | 177 | 188 |

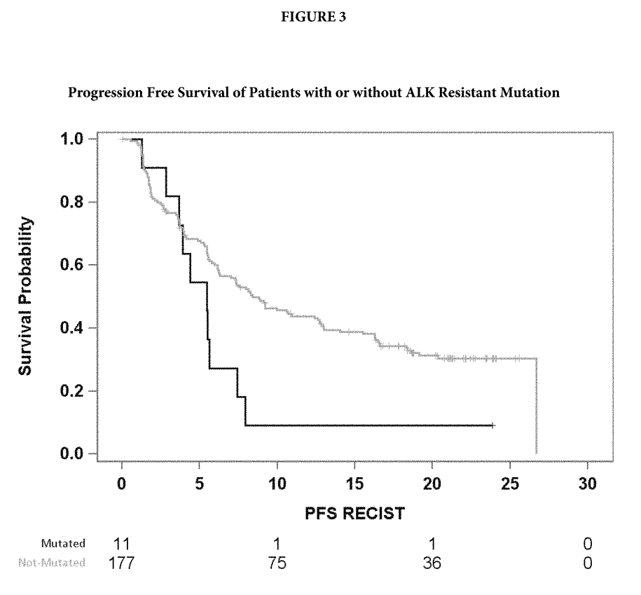

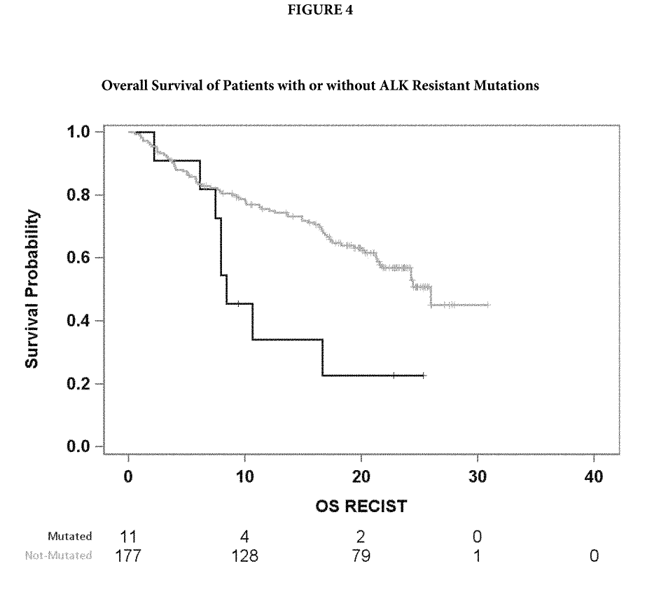

Progression free survival and overall survival rates were tracked for ALK resistant (ALKR) mutation positive (11) and negative (177) patients. As shown in FIGS. 3 and 4, survival rates were significantly lower in patients carrying a resistance mutation.

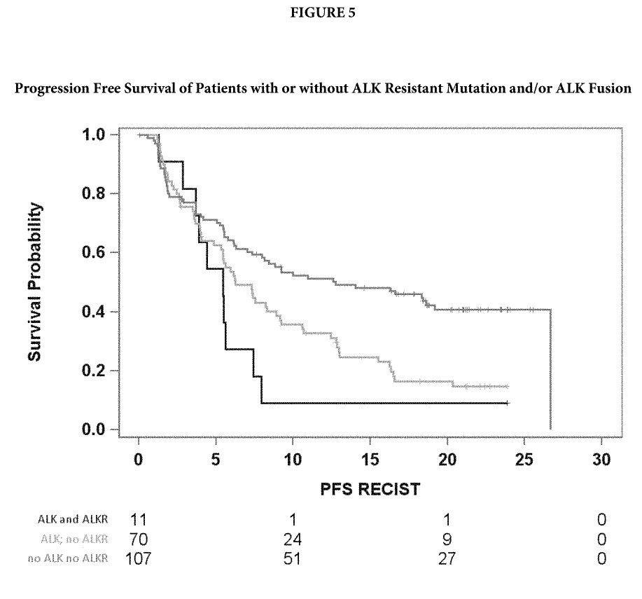

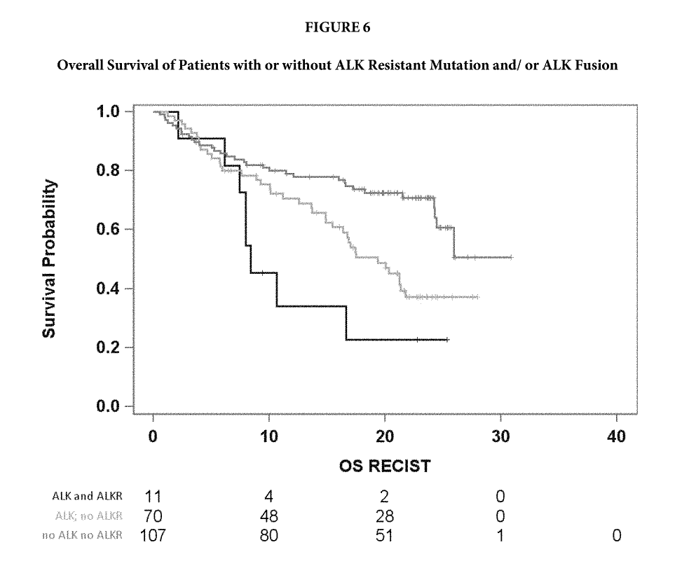

Progression free survival and overall survival rates were also tracked for (i) ALK fusion positive (ALK), ALK resistant mutation positive (ALKR) patients (11); (ii) ALK fusion positive, ALK resistant mutation negative patients (70); and (iii) ALK fusion negative, ALK resistant mutation negative patients (107). As shown in FIGS. 5 and 6, patients neither an ALK fusion or resistance mutation survived longest, followed by patents with an ALK fusion, but no ALK resistance mutation. Patients with both an ALK fusion and an ALK resistance mutation had the lowest survival rates.

### Example 4

### Effect of Treatment Duration and Number of ALK Resistance Mutations on Survival

Progression free survival and overall survival of patients with or without an ALK resistance mutation was correlated with the number of days on crizotinib first line treatment, followed by alectinib second line treatment. No significant correlation was found between the duration of crizotinib treatment and either progression free or overall survival.

### Example 5

### Effect of Number of ALK and Non-ALK Mutations on Survival

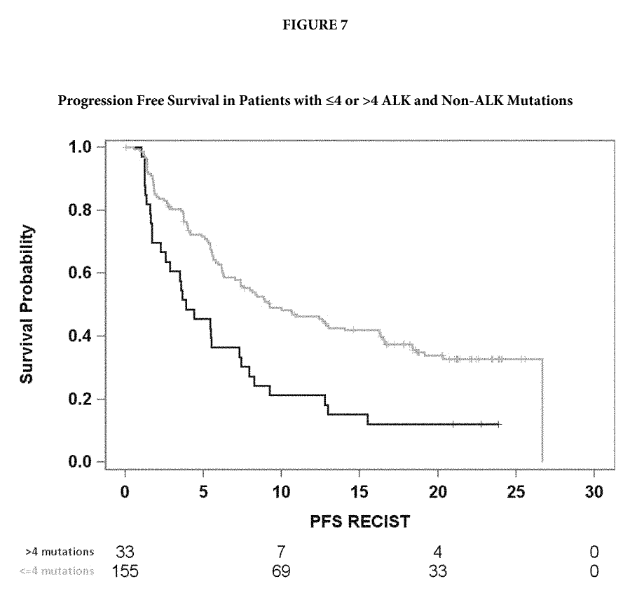

Progression free survival was tracked in patients with 4 or fewer ALK and non-ALK mutations and compared to those with more than 4 ALK and non-ALK mutations. These mutations include those shown in Table 2, Table 4, and Table 5. For non-ALK mutations, each gene was counted once, even if a patient had multiple mutations in that gene. Patients with fewer mutations had longer progression free survival, as shown in FIG. 7. The hazard ratio for having >4 mutations compared to 4 or fewer mutations was 2.12. The single nucleotide variant (SNV) ALK mutations detected include mutations that indicate susceptibility to treatment, resistance to treatment, and unknown significance for treatment. These are shown in Table 4. Table 5 shows additional ALK fusions, as well as non-ALK mutations. CNA indicates copy number amplification.

TABLE 4

| Susceptible | Resistant | Unknown |
|---|---|---|
| I1268V | R1209Q | M301I |
| S1206F/Y | G1202R | R1275Q |
| G1269A | I1171T/N/S | R1192P |
| L1196M/Q | T1151M | T1151R |
| C1156Y | G1202R | K507N/R |
| L1152R | V1180L | C1259R |
| F1174L/C/V | | I1268V |
| | | W1295C |
| | | I1322M |
| | | D1160A |
| | | I1057T |
| | | G940D |
| | | R1120W |
| | | S1281R |
| | | I1522M |
| | | A969D |
| | | A1200V |
| | | F1245L |
| | | R1212H |
| | | E1371K |
| | | P1213S |
| | | I248V |
| | | E1132K |

TABLE 5

| GENE | VARIANT | TYPE |
|---|---|---|
| DCDC2 | DCDC2 CNA | CNA |
| ALK | EML4-ALK E9;A20 | ALK-fusion |
| SLC17A8 | SLC17A8 CNA | CNA |
| INSL5 | INSL5 CNA | CNA |
| GRM8 | GRM8 CNA | CNA |
| PDGFRB | PDGFRB CNA | CNA |
| ERBB3 | ERBB3 CNA | CNA |
| CTNNB1 | CTNNB1 c.94G > T p.D32Y | SNV |
| ZIC4 | ZIC4 c.525G > T p.W175C | SNV |
| GRM8 | GRM8 c.1495G > C p.V499L | SNV |
| GRK7 | GRK7 c.1444G > A p.D482N | SNV |
| ERBB3 | ERBB3 c.974G > A p.G325E | SNV |
| JAK3 | JAK3 c.1993C > A p.P665T | SNV |
| KIT | KIT c.1757G > T p.R586I | SNV |
| KIT | KIT c.1758A > T p.R586S | SNV |
| TP53 | TP53 c.695T > A p.I232N | SNV |
| KIT | KIT c.2709G > C p.M903I | SNV |
| CPXCR1 | CPXCR1 c.674G > T p.G225V | SNV |
| JAK2 | JAK2 c.1849G > T p.V617F | SNV |
| KIT | KIT c.2307G > T p.L769F | SNV |
| ABL1 | ABL1 c.1249T > C p.S417P | SNV |
| NRAS | NRAS c.181C > A p.Q61K | SNV |
| SMAD4 | SMAD4 c.1081C > T p.R361C | SNV |
| JAK2 | JAK2 c.1849G > T p.V617F | SNV |
| TRIM58 | TRIM58 c.1254A > T p.E418D | SNV |
| ERBB3 | ERBB3 c.836A > T p.K279M | SNV |
| ARAF | ARAF c.641G > T p.R214L | SNV |
| KIT | KIT c.896C > G p.A299G | SNV |
| PIK3CA | PIK3CA c.3062A > G p.Y1021C | SNV |
| ALK | CCDC142-ALK | ALK-fusion |
| other | EML4-CCDC142 | Fusion |
| SPTA1 | SPTA1 c.2671C > T p.R891* | SNV |
| IDH1 | IDH1 c.401C > A p.A134D | SNV |
| ROBO2 | ROBO2 c.280C > T p.R94C | SNV |
| EPHX4 | EPHX4 CNA | CNA |
| GRM8 | GRM8 CNA | CNA |
| INSL5 | INSL5 CNA | CNA |
| other | CD74-RABGAP1L | Fusion |
| MET | MET c.3803T > C p.M1268T | SNV |
| ATM | ATM c.7202T > C p.I2401T | SNV |
| ROS1 | ROS1 c.5109A > T p.K1703N | SNV |
| TP53 | TP53 c.736A > G p.M246V | SNV |
| MET | MET c.3583C > G p.L1195V | SNV |
| DDR2 | DDR2 c.1754T > A p.M585K | SNV |
| PGM5 | PGM5 c.1421C > G p.A474G | SNV |
| TP53 | TP53 c.743G > A p.R248Q | SNV |
| MET | MET Exon14del | Indel |
| ALK | STRN-ALK | ALK-fusion |
| KIF5B | KIF5B CNA | CNA |
| ATM | ATM CNA | CNA |
| KIT | KIT CNA | CNA |
| CNBD1 | CNBD1 CNA | CNA |
| C6 | C6 CNA | CNA |
| CWF19L2 | CWF19L2 CNA | CNA |
| TNR | TNR c.682G > T p.D228Y | SNV |
| KRAS | KRAS c.37G > T p.G13C | SNV |
| KIF5B | KIF5B CNA | CNA |
| EML4 | EML4 c.1339C > T p.Q447* | SNV |
| ERBB3 | ERBB3 c.303C > A p.N101K | SNV |
| TP53 | TP53 c.574C > T p.Q192* | SNV |
| SMAD4 | SMAD4 c.1028C > A p.S343* | SNV |
| TP53 | TP53 c.886G > A p.D296N | SNV |
| MSN | MSN c.1109G > A p.R370H | SNV |
| ROS1 | ROS1 c.5735T > A p.L1912Q | SNV |
| TP53 | TP53 c.202G > T p.E68* | SNV |
| TP53 | TP53 c.623A > T p.D208V | SNV |
| EML4 | EML4 c.2668G > A p.E890K | SNV |
| FGFR2 | FGFR2 c.1226C > T p.A409V | SNV |
| ESR1 | ESR1 c.1688C > T p.T563M | SNV |
| VHL | VHL c.241C > T p.P81S | SNV |
| NRAS | NRAS c.350A > T p.K117M | SNV |
| TP53 | TP53 c.702C > A p.Y234* | SNV |
| KIT | KIT c.335G > C p.R112T | SNV |
| other | EML4-LBH | Fusion |
| ALK | MYO7A-ALK | ALK-fusion |
| POM121L12 | POM121L12 c.481G > A | SNV |
| | p.A161T | |
| TP53 | TP53 c.916C > T p.R306* | SNV |
| MLH1 | MLH1 c.1069G > T p.G357W | SNV |
| SYT4 | SYT4 c.113G > T p.C38F | SNV |
| KRAS | KRAS c.183A > T p.Q61H | SNV |
| CSMD3 | CSMD3 c.2144C > A p.P715H | SNV |
| TP53 | TP53 c.527G > T p.C176F | SNV |
| APC | APC c.2129T > C p.L710P | SNV |
| RAF1 | RAF1 c.1193G > T p.R398L | SNV |
| CDH9 | CDH9 c.2361C > A p.D787E | SNV |
| TP53 | TP53 c.991C > T p.Q331* | SNV |
| CSMD3 | CSM D3 c.9464G > A p.C3155Y | SNV |
| GNAS | GNAS c.601C > T p.R201C | SNV |
| MET | MET c.4087G > A p.A1363T | SNV |
| JAK2 | JAK2 c.2617G > C p.D873H | SNV |
| GNAS | GNAS c.601C > T p.R201C | SNV |
| CDKN2A | CDKN2A c.149A > C p.Q50P | SNV |
| CSMD3 | CSMD3 c.6915C > A p.N2305K | SNV |
| KIT | KIT c.2341G > T p.A781S | SNV |
| TP53 | TP53 c.989T > G p.L330R | SNV |
| MAP2K1 | MAP2K1 c.102G > T p.Q34H | SNV |
| CWF19L2 | CWF19L2 CNA | CNA |
| FGFR1 | FGFR1 CNA | CNA |
| ATP2B4 | ATP2B4 c.212C > T p.A71V | SNV |
| NRXN1 | NRXN1 c.2594T > G p.F865C | SNV |
| ATM | ATM c.8512A > G p.K2838E | SNV |
| KRAS | KRAS c.40G > A p.V14I | SNV |
| RET | RET c.2225C > T p.T742M | SNV |
| other | EML4-RABGAP1L | Fusion |
| other | CD74-ROS1 | Fusion |
| HCN1 | HCN1 c.2503G > C p.V835L | SNV |
| PIK3CA | PIK3CA c.35G > A p.G12D | SNV |
| TP53 | TP53 c.833C > G p.P278R | SNV |
| FBXW7 | FBXW7 c.1448T > C p.L483P | SNV |
| TP53 | TP53 c.574C > G p.Q192E | SNV |
| ALK | RAD51AP2-ALK | ALK-fusion |
| other | EM L4-AKAP13 | Fusion |
| IDH1 | IDH1 c.394C > T p.R132C | SNV |
| MET | MET c.3979C > T p.R1327C | SNV |
| TP53 | TP53 c.658T > C p.Y220H | SNV |
| UNC5C | UNC5C c.1495T > A p.S499T | SNV |
| CTNNB1 | CTNNB1 c.110C > T p.S37F | SNV |
| TP53 | TP53 c.380C > T p.S127F | SNV |
| NPM1 | NPM1 CNA | CNA |
| AKT1 | AKT1 CNA | CNA |
| SLC5A10 | SLC5A10 CNA | CNA |
| IDH2 | IDH2 CNA | CNA |
| SLC17A8 | SLC17A8 CNA | CNA |
| FGFR1 | FGFR1 CNA | CNA |
| PDGFRB | PDGFRB CNA | CNA |
| KIT | KIT CNA | CNA |
| ERBB3 | ERBB3 CNA | CNA |
| FGFR2 | FGFR2 CNA | CNA |
| MAP2K1 | MAP2K1 CNA | CNA |
| SLPI | SLPI CNA | CNA |
| TP53 | TP53 CNA | CNA |
| CWF19L2 | CWF19L2 CNA | CNA |
| ERBB2 | ERBB2 CNA | CNA |
| CCND1 | CCND1 CNA | CNA |
| TRIM58 | TRIM58 c.1433C > T p.A478V | SNV |
| ZIC4 | ZIC4 c.531G > T p.E177D | SNV |
| CNTNAP2 | CNTNAP2 c.1138G > T | SNV |
| | p.A380S | |
| TP53 | TP53 c.452C > A p.P151H | SNV |
| KRAS | KRAS c.183A > C p.Q61H | SNV |
| JAK3 | JAK3 c.2896G > T p.A966S | SNV |
| TP53 | TP53 c.832C > G p.P278A | SNV |
| ST6GAL2 | ST6GAL2 c.1544A > T p.Q515L | SNV |
| KRAS | KRAS c.35G > T p.G12V | SNV |
| GATA3 | GATA3 c.1082G > C p.G361A | SNV |
| IDH2 | IDH2 c.514A > G p.R172G | SNV |
| CPXCR1 | CPXCR1 c.755G > A p.G252D | SNV |
| GRM8 | GRM8 c.1820G > A p.R607H | SNV |
| VHL | VHL Deletion | Indel |
| CPXCR1 | CPXCR1 c.365A > T p.N122I | SNV |
| TP53 | TP53 c.817C > T p.R273C | SNV |
| LRIG3 | LRIG3 c.2779G > T p.G927C | SNV |
| CSMD3 | CSMD3 c.2210A > G p.Y737C | SNV |
| ERBB2 | ERBB2 c.2720A > G p.K907R | SNV |
| TP53 | TP53 c.524G > A p.R175H | SNV |
| POM121L12 | POM121L12 c.141G > T | SNV |
| | p.Q47H | |
| CTNNB1 | CTNNB1 c.94G > C p.D32H | SNV |
| FERD3L | FERD3L CNA | CNA |
| IDH2 | IDH2 CNA | CNA |
| ERBB3 | ERBB3 CNA | CNA |
| KIF5B | KIF5B CNA | CNA |
| CCND2 | CCND2 CNA | CNA |
| GNA11 | GNA11 CNA | CNA |
| KRAS | KRAS CNA | CNA |
| ALK | DCTN1-ALK | ALK-fusion |
| KIT | KIT c.978C > G p.N326K | SNV |
| TRHDE | TRHDE c.973C > A p.P325T | SNV |
| ATM | ATM c.9023G > A p.R3008H | SNV |
| MLH1 | MLH1 c.1232T > G p.I411S | SNV |
| ROBO2 | ROBO2 c.3254C > T p.T1085M | SNV |
| FGFR2 | FGFR2 c.1675G > A p.A559T | SNV |
| ABL1 | ABL1 c.1165A > G p.T389A | SNV |
| TP53 | TP53 c.455C > G p.P152R | SNV |
| CDH9 | CDH9 c.2318G > C p.R773P | SNV |
| TP53 | TP53 c.853G > A p.E285K | SNV |
| DCDC1 | DCDC1 c.801G > T p.L267F | SNV |
| CDKN2A | CDKN2A c.35C > T p.S12L | SNV |
| TP53 | TP53 c.517G > T p.V173L | SNV |
| SATB2 | SATB2 c.1429G > T p.E477* | SNV |
| HCN1 | HCN1 c.2549T > C p.I850T | SNV |
| SLPI | SLPI c.309T > G p.N103K | SNV |
| VHL | VHL Deletion | Indel |
| other | ETV6-ROS1 | Fusion |
| KCNB2 | KCNB2 c.338A > T p.E113V | SNV |
| SLC34A2 | SLC34A2 c.285C > G p.F95L | SNV |
| CWF19L2 | CWF19L2 CNA | CNA |
| other | EML4-AFF3 | Fusion |
| PTPRD | PTPRD c.3631T > C p.Y1211H | SNV |
| SLC34A2 | SLC34A2 c.1462G > A p.A488T | SNV |
| EPHA6 | EPHA6 c.857G > C p.G286A | SNV |
| ATM | ATM c.8495G > A p.R2832H | SNV |
| EML4 | EML4 c.2668G > A p.E890K | SNV |
| GNAS | GNAS c.2524C > T p.R842C | SNV |
| FGFR1 | FGFR1 c.1520G > A p.R507H | SNV |
| REG3A | REG3A c.455G > T p.S152I | SNV |
| CSMD3 | CSMD3 c.10859C > G | SNV |
| | p.S3620* | |
| ZIC4 | ZIC4 c.137T > A p.L46H | SNV |
| LRIG3 | LRIG3 c.2768T > G p.L923W | SNV |
| DDR2 | DDR2 c.2063A > C p.K688T | SNV |
| POM121L12 | POM121L12 c.418G > A | SNV |
| | p.G140R | |
| PNPLA1 | PNPLA1 c.175G > A p.V59I | SNV |
| ROS1 | ROS1 c.5131G > T p.A1711S | SNV |
| CCND1 | CCND1 CNA | CNA |
| TP53 | TP53 c.637C > T p.R213* | SNV |
| TP53 | TP53 c.745A > T p.R249W | SNV |
| SMAD4 | SMAD4 c.1049T > A p.V350D | SNV |
| ALK | EML4-ALK E6;A16 | ALK-fusion |
| ALK | EML4-ALK E6;A19 | ALK-fusion |
| TP53 | TP53 c.653T > G p.V218G | SNV |
| CSMD3 | CSMD3 c.9029A > G p.K3010R | SNV |
| GRM8 | GRM8 c.1820G > A p.R607H | SNV |
| JAK2 | JAK2 c.1849G > T p.V617F | SNV |
| ADAMTS5 | ADAMTS5 c.1437G > T | SNV |
| | p.Q479H | |
| TP53 | TP53 c.536A > G p.H179R | SNV |
| CTNNB1 | CTNNB1 c.101G > T p.G34V | SNV |
| TP53 | TP53 c.589G > C p.V197L | SNV |
| ESR1 | ESR1 c.775C > G p.R259G | SNV |
| MAP2K1 | MAP2K1 c.412G > A p.E138K | SNV |
| LRP1B | LRP1B c.1065C > A p.D355E | SNV |
| KRAS | KRAS c.34G > T p.G12C | SNV |
| LRRIQ3 | LRRIQ3 c.1490C > A p.A497D | SNV |
| EPHA6 | EPHA6 c.1099G > A p.E367K | SNV |
| TP53 | TP53 c.526T > A p.C176S | SNV |
| ALK | EML4-ALK E17;A20 | ALK-fusion |
| HCN1 | HCN1 c.2549T > C p.I850T | SNV |
| ERBB3 | ERBB3 c.2821T > A p.Y941N | SNV |
| GRM8 | GRM8 CNA | CNA |
| TNR | TNR CNA | CNA |
| SLC17A8 | SLC17A8 CNA | CNA |
| INSL5 | INSL5 CNA | CNA |
| DUSP22 | DUSP22 c.138G > C p.W46C | SNV |
| SLC34A2 | SLC34A2 c.1717C > T p.R573* | SNV |
| ZNF598 | ZNF598 c.1491G > T p.E497D | SNV |
| TP53 | TP53 c.659A > G p.Y220C | SNV |
| INSL5 | INSL5 CNA | CNA |
| GRM8 | GRM8 CNA | CNA |
| MYD88 | MYD88 CNA | CNA |
| ESR1 | ESR1 CNA | CNA |
| DIO2 | DIO2 CNA | CNA |
| TNR | TNR CNA | CNA |
| EXOC4 | EXOC4 CNA | CNA |
| FGFR1 | FGFR1 CNA | CNA |
| AR | AR CNA | CNA |
| PDGFRB | PDGFRB CNA | CNA |
| FERD3L | FERD3L CNA | CNA |
| SLPI | SLPI CNA | CNA |
| other | EML4-CUX1 | Fusion |
| CTNNB1 | CTNNB1 c.110C > A p.S37Y | SNV |
| ATM | ATM c.7213A > G p.M2405V | SNV |
| DUSP22 | DUSP22 c.247C > T p.R83W | SNV |
| ALK | EML4-ALK E13;A20 | ALK-fusion |
| TP53 | TP53 c.911C > T p.T304I | SNV |
| EGFR | EGFR c.2591C > T p.A864V | SNV |

While the invention has been described in detail with reference to specific examples, it will be apparent to one skilled in the art that various modifications can be made within the scope of this invention. The scope of the invention should not be limited by the examples described herein. All patents, publications, websites, Genbank (or other database) entries disclosed herein are incorporated by reference in their entireties.

1. A method of treating a patient having a tumor possibly harboring cells with a mutation in anaplastic lymphoma kinase (ALK) gene, comprising:
(a) testing a sample from the patient for the presence of ALK mutations R1209Q (G3636A) and/or I1268V (A3802G), and
(b) if the mutation R1209Q is present, not administering an ALK inhibitor compound to the patient, or if the patient is already receiving an ALK inhibitor compound, administering an alternative ALK inhibitor compound; and
(c) if the mutation I1268V is present or no mutations are present, administering an ALK inhibitor compound to the patient.

2. The method of claim 1, wherein the ALK inhibitor compound or alternative ALK inhibitor compound is selected from the group consisting of alectinib, crizotinib, ceritinib, alectinib, brigatinib, lorlatinib, and entrectinib.

3. The method of claim 1, wherein the testing is performed using an oligonucleotide complementary to the mutant sequence.

4. The method of claim 1, wherein the testing is performed by allele-specific PCR or real-time PCR with an allele-specific probe.

5. The method of claim 1, further comprising testing the sample from the patient for the presence of one or more of the ALK mutations selected from the group consisting of L1152R, C1156Y, F1174L, L1196M, G1269A, and G1548E and if any of the mutations is present, not administering an ALK inhibitor compound to the patient, or if the patient is already receiving an ALK inhibitor compound, administering an alternative ALK inhibitor compound.

6. The method of claim 1, comprising:
testing the sample from the patient for the presence of one or more ALK fusion products;
if one or more ALK fusion products is present, administering an ALK inhibitor compound to the patient; and
if one or more ALK fusion products and one or more mutations selected from the group consisting of R1209Q, L1152R, C1156Y, F1174L, L1196M, G1269A, and G1548E is present, not administering an ALK inhibitor compound to the patient, or if the patient is already receiving an ALK inhibitor compound, administering an alternative ALK inhibitor compound.

7. A method for determining the likelihood of response of a cancer patient to an ALK inhibitor compound comprising: testing a sample from the patient for ALK mutations R 1209Q and/or I1268V and, if the mutation R1209Q is present, reporting that the patient will likely not respond to the ALK inhibitor compound and if the mutation I1268V is present or no mutation is present, reporting that the patient will likely respond to the ALK inhibitor compound.

8. The method of claim 7, wherein said ALK inhibitor compound is selected from the group consisting of alectinib, crizotinib, ceritinib, alectinib, brigatinib, lorlatinib, and trectinib.

9. The method of claim 7, wherein the testing is performed using an oligonucleotide complementary to the mutant sequence.

10. The method of claim 7, wherein the testing is performed by allele-specific PCR or real-time PCR with an allele-specific probe.

11. The method of claim 7, further comprising testing the sample for the presence of one or more of ALK mutations selected from the group consisting of L1152R, C1156Y, F1174L, L1196M, G1269A, and G1548E, and if any of the mutations is present, reporting that the patient is not likely to respond to the ALK inhibitor compound, or if the patient is already receiving an ALK inhibitor compound, administering an alternative ALK inhibitor compound.

12. The method of claim 7, further comprising:
testing the sample from the patient for the presence of one or more ALK fusion products;
if one or more ALK fusion products is present, administering an ALK inhibitor compound to the patient; and
if one or more ALK fusion products and one or more mutations selected from the group consisting of R1209Q, L1152R, C1156Y, F1174L, L1196M, G1269A, and G1548E is present, not administering an ALK inhibitor compound to the patient, or if the patient is already receiving an ALK inhibitor compound, administering an alternative ALK inhibitor compound.

13. A method for selecting anaplastic lymphoma kinase (ALK) inhibitor therapy for a patient having a tumor harboring cells with a mutation in the gene who has been previously treated with alectinib, comprising:
(a) testing a sample from the patient for the presence of ALK mutations R1209Q (G3636A) and/ or I1268V (A3802G), and
(b) if the mutation R1209Q is present, not administering to the patient an ALK inhibitor compound or administering an alternative ALK inhibitor compound, and
(c) if the mutation I1268V is present or no mutations are present, administering alectinib to the patient.

14. The method of claim 13, further comprising testing the sample for the presence of one or more of the ALK mutations L1152R, C1156Y, F1174L, L1196M, G1269A, and G1548E and if any of the mutations is present, not administering to the patient the ALK inhibitor compound or administering an alternative ALK inhibitor compound.

15. The method of claim 13, wherein the alternative ALK inhibitor compound is selected from the group consisting of crizotinib, ceritinib, brigatinib, lorlatinib and entrectinib.

16. The method of claim 13, further comprising testing the sample from the patient for the presence of an ALK fusion product and if an ALK fusion product is present, administering an ALK inhibitor compound to the patient.

17. A kit for detecting mutations in the ALK gene comprising oligonucleotides that specifically bind to mutations R1209Q and I1268V in the ALK gene.

18. The kit of claim 17, further comprising nucleic acid precursors, nucleic acid polymerase and reagents and solutions necessary to support the activity of the nucleic acid polymerase.

19. The kit of claim 17, further comprising one or more oligonudeotides that specifically hybridize to one or more ALK mutations L1152R, C1156Y, F1174L, L1196M, G1269A, and G1548E.

20. The kit of claim 17, further comprising one or more oligonudeotides that specifically detect an ALK fusion product.

21. A method of treating a patient having a tumor possibly harboring cells with a mutation in anaplastic lymphoma kinase (ALK) gene, comprising:
(a) testing a sample from the patient for the presence of one or more ALK fusion products;
(b) if the one or more ALK fusion products tested in (a) is present, administering an ALK inhibitor compound;
(c) testing a sample from the patient for the presence of one or more ALK resistance mutations selected from the group consisting of R1209Q, L1152R, C1156Y, F1174L, L1196M, G1269A, and G1548E; and
(d) if the one or more ALK resistance mutations tested in (c) is present, not administering an ALK inhibitor compound to the patient, or if the patient is already receiving an ALK inhibitor compound, administering an alternative ALK inhibitor compound,
wherein if both the one or more ALK fusion products and one or more ALK resistance mutation are present, not administering an ALK inhibitor compound to the patient, or if the patient is already receiving an ALK inhibitor compound, administering an alternative ALK inhibitor compound.

22. The method of claim 21, wherein the ALK inhibitor compound or alternative ALK inhibitor compound is selected from the group consisting of alectinib, crizotinib, centinib, brigatinib, lorlatinib, and entrectinib.
